(19) United States
(12) Patent Application Publication
(10) Pub. No.: US 2023/0157884 A1
(43) Pub. Date: May 25, 2023

(54) IMAGE-GUIDED LASER BEAM AIM TO TREAT VITREOUS FLOATERS

(71) Applicant: Alcon Inc., Fribourg (CH)

(72) Inventor: Zsolt Bor, San Clemente, CA (US)

(21) Appl. No.: 18/050,370

(22) Filed: Oct. 27, 2022

Related U.S. Application Data
(60) Provisional application No. 63/281,504, filed on Nov. 19, 2021.

(51) Int. Cl. A61F 9/008

(52) U.S. Cl. CPC A61F 9/00814; A61F 2009/00861; A61F 2009/00897; A61N 2005/105

(57) ABSTRACT

In certain embodiments, an ophthalmic laser surgical system for imaging and treating a target in an eye includes a laser device, imaging system, and computer. The laser device directs the focus of a laser beam towards an intended location (x0, y0, z0) of the target to yield a cavitation bubble in the vitreous. The imaging system directs imaging beams towards the target, receives the imaging beams reflected from the eye, generates an image of the cavitation bubble from the reflected imaging beams, and measures an actual location (x, y, z) of the cavitation bubble according to the image. The computer determines an error vector that describes an error between the intended location and the actual location, determines a correction vector to compensate for the error, and instructs the laser device to use the correction vector to direct the laser beam towards the target to treat the target.

### TECHNICAL FIELD

The present disclosure relates generally to ophthalmic laser surgical systems, and more particularly to improving image-guided laser beam aim to treat vitreous floaters.

### BACKGROUND

In ophthalmic laser surgery, a surgeon may direct a laser beam into the eye to treat the eye. For example, a laser beam may be directed into the vitreous to treat eye floaters. Eye floaters are clumps of collagen proteins that form in the vitreous. These clumps can disturb vision with moving shadows and distortions. The laser beam may be used to fragment floaters to improve vision.

### BRIEF SUMMARY

In certain embodiments, an ophthalmic laser surgical system for imaging and treating a target in an eye includes a laser device, an imaging system, and a computer. The eye has an eye axis that defines a z-axis, which defines xy-planes. An xyz-location is relative to an xy-plane and the z-axis. The laser device directs a focus of a laser beam along a laser beam path towards an intended location (x0, y0, z0) of the target in a vitreous of the eye to yield a cavitation bubble in the vitreous. The imaging system directs one or more imaging beams along an imaging beam path towards the target, receives the imaging beams reflected from the eye, generates an image of the cavitation bubble from the reflected imaging beams, and measures an actual location (x, y, z) of the cavitation bubble according to the image. The computer determines an error vector that describes an error between the intended location and the actual location, determines a correction vector to compensate for the error, and instructs the laser device to use the correction vector to compensate for the error to direct the laser beam towards the target to treat the target.

Embodiments may include none, one, some, or all of the following features:

The target comprises an eye floater.

The ophthalmic laser surgical system includes an xy-scanner that: receives the imaging beams from the imaging system and directs the imaging beams along the imaging beam path towards the target; and receives the laser beam from the laser device and directs the laser beam along the laser beam path aligned with the imaging beam path towards the target to treat the target.

The error vector is (x−x0, y−y0, z−z0).

The correction vector is (x0−x, y0−y, z0−z).

The imaging system includes an xy-imaging device configured to provide an xy-location of the target relative to an xy-plane, and/or a z-imaging device configured to provide a z-location of the target relative to the z-axis.

The imaging system includes an xyz-imaging device configured to provide the xyz-location of the target.

In certain embodiments, a method for imaging and treating a target in an eye includes directing, by a laser device, the focus of a laser beam along a laser beam path towards an intended location (x0, y0, z0) of the target in the vitreous of the eye to yield a cavitation bubble in the vitreous. The eye has an eye axis that defines a z-axis, which in turn defines xy-planes. An xyz-location is relative to an xy-plane and the z-axis. Imaging beams are directed along an imaging beam path towards the target by an imaging system. The imaging beams reflected from the eye are received by the imaging system, which generates an image of the cavitation bubble from the reflected imaging beams. The actual location (x, y, z) of the cavitation bubble is measured by the imaging system according to the image. An error vector that describes the error between the intended location and the actual location is determined by a computer, which determines a correction vector to compensate for the error. The laser device is instructed by the computer to use the correction vector to compensate for the error to direct the laser beam towards the target to treat the target.

Embodiments may include none, one, some, or all of the following features:

The target comprises an eye floater.

The method further comprises: receiving, by an xy-scanner, the one or more imaging beams from the imaging system and directing the one or more imaging beams along the imaging beam path towards the target; and receiving, by the xy-scanner, the laser beam from the laser device and directing the laser beam along the laser beam path aligned with the imaging beam path towards the target to treat the target.

The error vector comprises (x−x0, y−y0, z−z0).

The correction vector comprises (x0−x, y0−y, z0−z).

The method further comprises providing, by an xy-imaging device of the imaging system, an xy-location of the target relative to an xy-plane.

The method further comprises providing, by a z-imaging device of the imaging system, a z-location of the target relative to the z-axis.

The method further comprises providing, by an xyz-imaging device of the imaging system, the xyz-location of the target.

### BRIEF DESCRIPTION OF THE DRAWINGS

FIG. 1 illustrates an example of an ophthalmic laser surgical system that may be used to treat an eye, according to certain embodiments; and

FIGS. 2 and 3 illustrate an example of a method for improving laser beam aiming that may be used by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Accurate laser beam aim is important when treating eye floaters to avoid damaging healthy issue and overexposing the retina. However, the aiming accuracy may be affected by several factors. A major source of error is the variation of corneal refractive power of different patient eyes, which is approximately 43+/−1.5 diopters. This results in approximately +/−620 um depth error in the focus of the laser beam. Other factors include corneal folds, dynamic errors of laser and imaging scanners, co-calibration error between laser and imaging beams, and self-focusing of the laser beam.

Accordingly, in certain embodiments, an ophthalmic laser surgical system improves aiming accuracy by determining an aiming error and compensating for the error.

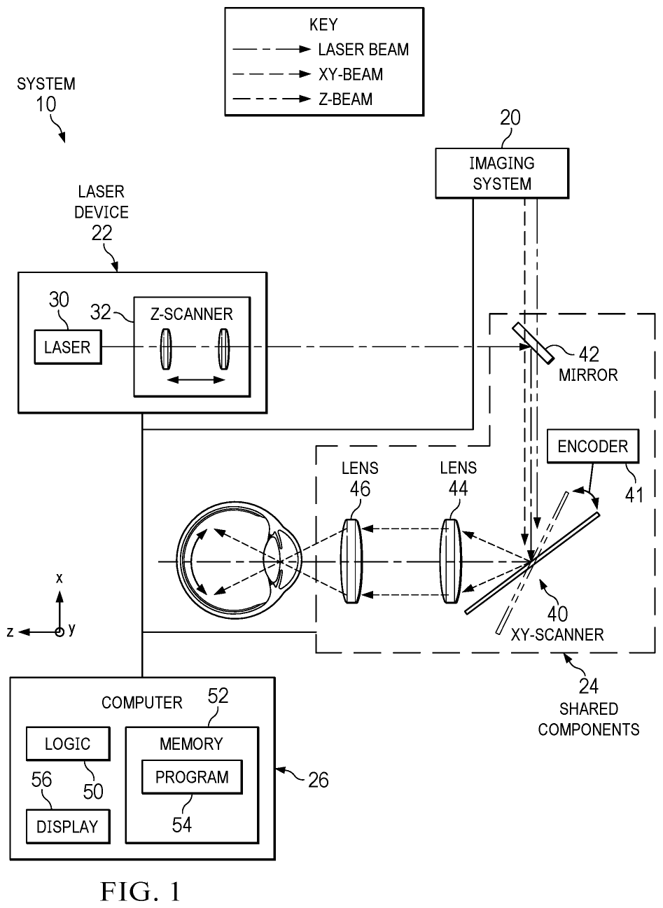

FIG. 1 illustrates an example of an ophthalmic laser surgical system 10 that may be used to treat an eye, according to certain embodiments. In the example, the eye has an eye axis (e.g., a visual or optical axis), which defines a z-axis. In turn, the z-axis defines xy-planes orthogonal to the z-axis. An xyz-location may include an xy-location and a z-location, where the xy-location is relative to an xy-plane, and the z-location is relative to the z-axis.

As an overview, system 10 includes an imaging system 20, a laser device 22, one or more shared components 24, and a computer 26, coupled as shown. Laser device 22 includes a laser 30 and a z-scanner 32, coupled as shown. Shared components 24 include an xy-scanner 40, an xy-encoder 41, and optical elements (such as a mirror 42 and lenses 44 and 46), coupled as shown. Computer 26 includes logic 50, a memory 52 (which stores a computer program 54), and a display 56, coupled as shown.

As an overview of operation of system 10, laser device 22 directs a focus of a laser beam along a laser beam path towards an intended location (x0, y0, z0) of the target in a vitreous of the eye to yield a cavitation bubble in the vitreous. Imaging system 20 directs one or more imaging beams along an imaging beam path towards the target, receives the imaging beams reflected from the eye, generates an image of the cavitation bubble from the reflected imaging beams, and measures an actual location (x, y, z) of the cavitation bubble according to the image. Computer 26 determines an error vector that describes an error between the intended location and the actual location, determines a correction vector to compensate for the error, and instructs the laser device to use the correction vector to compensate for the error to direct the laser beam towards the target to treat the target.

Turning to the parts of the system, imaging system 20 includes one or more imaging device(s) that image a target in the vitreous. To image the target, an imaging device directs an imaging beam along an imaging beam path towards a target within the eye. The target reflects the imaging beam, and the device detects the reflected light and generates image(s) from the reflected beam, which may be displayed on display 56. In certain embodiments, the images are displayed in real time as a video. The devices may utilize the same or different technologies (e.g., scanning laser ophthalmoscopy (SLO) and/or interferometry). The imaging device(s) may also provide the x, y, and/or z locations of the target, in any suitable manner. For example, one device may provide the x, y, and z locations. As another example, one device may provide the x and y locations, while another may provide the z location.

In certain embodiments, imaging system 20 may include an xy-imaging device and a z-imaging device. The xy-imaging device provides the xy-location of the target, and the z-imaging device provides the z-location of the target. Examples of an xy-imaging device include a scanning laser ophthalmoscopy (SLO) device and a fundus camera. Examples of a z-imaging device include an interferometer device and a scanning confocal ophthalmoscope camera. In other embodiments, imaging system 20 may be an xyz-imaging device configured to provide the xy-location and z-location of the target. An example of an xyz-imaging device includes an interferometer device.

In certain embodiments, imaging system 20 may include an SLO device. In the embodiments, the SLO device provides the xy-location of a floater, which may be given by the xy-location of the shadow of the floater on the retina, such that the shadow xy-location may be used as the floater xy-location. In certain embodiments, the SLO device has a higher frame rate than that of an interferometer device, so may provide better xy-tracking of a target such as a floater. The SLO device may have any suitable frame rate, e.g., 30 to 60, such 45 to 55, or approximately 50, frames per sec (frames/s).

In certain embodiments, imaging system 20 may include an interferometer device with any suitable interferometer, e.g., a Fourier domain type (such as a swept source or a spectral domain type) that utilizes a fast Fourier transform (FFT). Examples of interferometer devices include an optical coherence tomography (OCT) device (such as a swept-source OCT device) and a swept source A-scan interferometer (SSASI) device. A SASSI device performs only A-scans.

As an example of operation of an interferometer device, a splitter splits the light into measurement light and reference light. The reference light is directed to a reference arm system. The measurement light is directed through shared components 24 towards a point of the eye. The interferometer device may direct the beam to different z-locations with, e.g., an electrically tunable lens (e.g., Optotune lens) or aspheric lens. The measurement light penetrates the eye in the z-direction and is reflected by the internal portion of the eye. The reflected measurement light provides information about the internal portion in the z-direction. For example, the light may indicate the location of the surfaces (e.g., the anterior and/or posterior surfaces) of a floater, the lens (natural or intraocular lens (IOL)), and/or the retina. Accordingly, the light may indicate the z-location and thickness of the floater in the z-direction.

The reflected measurement light travels back through the same path. The splitter combines the reflected measurement light with reference light from the reference arm system to yield interference signal light. A detector detects the interference signal light and aggregates the photon reflections in the z-direction to yield an A-scan, i.e., the reflection intensity distribution of the measurement light in the z-direction. Additional A-scans may be performed in another direction (e.g., the x- or y-direction) generate multiple adjacent A-scans, which may be compiled into a two-dimensional B-scan. The multiple A-scans may be performed at any suitable rate, e.g., once every 10 to 30 ms, such as every approximately 20 ms, to determine the z-location of the floater.

In certain embodiments, the interferometer device generates scans to determine the location and shape of a floater in the x- and z-directions. For example, the interferometer device generates an A-scan in the z-direction to yield information about the target in the z-direction. The shared xy-scanner then moves the beam in the x-direction to generate another z-direction A-scan, and so on, in order to yield multiple z-direction A-scans that are adjacent to each other in the x-direction (or y-direction), which provides information about the target in the x-direction (or y-direction). There may be any suitable distance between adjacent A-scans, e.g., 30 to 60 microns, such as 50 microns. Xy-encoder 41 reports the xy-location of the imaging beam as the beam is scanned.

In certain embodiments, the interferometer device may be a long-range interferometer system, which can image a z-direction range as long as 24 to 30 mm, such as 27 mm, e.g., the length of the optical axis of the eye. The interferometer device may be a full depth interferometer system, which can image at least the depth of the posterior chamber of the eye. In other embodiments, the interferometer device may have multiple reference arms, each arm having a different range in the z-direction.

Turning to laser device 22, laser 30 generates a laser beam with any suitable wavelength, e.g., in a range from 400 nm to 2000 nm. Laser device 22 delivers laser pulses at any suitable repetition rate (e.g., 30 to 120 kilohertz (kHz)), such as a high repetition rate of 100 kHz or greater. A laser pulse may have any suitable pulse duration (e.g., in the picosecond to nanosecond range, such as 20 fs to 1000 ns), any suitable pulse energy (e.g., 1 microjoule (P) to 1 millijoule (mJ)), and a focal point of any suitable size (e.g., 3 to 10 microns (μm), such as 7 μm).

Z-scanner 32 longitudinally directs the focal point of the laser beam to a specific location in the z-direction. Examples of z-scanner 32 include a longitudinally adjustable lens, a lens of variable refractive power, an electrically or mechanically tunable lens (e.g., Optotune lens), an electrically or mechanically tunable telescope, or a deformable mirror that can control the z-location of the focal point. Z-scanner 32 may direct the focal point in any suitable manner. In certain embodiments, z-scanner 32 receives the z-location of the target from imaging system 20 (and may receive it via computer 26), and directs the laser beam towards the z-location of the target. In certain embodiments, laser device 22 may also include a fast xy-scanner used in tandem with z-scanner 32 to, e.g., create a 3D pulse pattern. Examples of such scanners include a resonant scanner or acousto optical scanner.

Shared components 24 direct imaging and laser beams from imaging system 20 and laser device 22, respectively, towards the eye. Because imaging and laser beams both use shared components 24, both beams are affected by the same optical distortions (e.g., fan distortion of scanners, barrel or pillow distortions of the scanner lens, refractive distortions from the inner eye surfaces, and other distortions). The distortions affect both beams in the same way, so the distortions are compensated for. This allows for aiming the laser beam using images generated by the imaging beam with improved accuracy.

As an overview of operation of shared components 24, mirror 42 directs a beam (imaging and/or laser beam) towards xy-scanner 40, which transversely directs the beam towards lens 44. Lenses 44 and 46 direct the beam towards eye. Shared components 24 may also provide spectral and polarization coupling and decoupling of imaging and laser beams to allow the beams to share the same path.

Turning to the details of shared components 24, xy-scanner 40 transversely directs the focal point of the beam in the x- and y-directions. Xy-scanner 40 changes the angle of incidence of the beam into the pupil, allowing for the beam to cover a wider range within the eye. Xy-scanner 40 may transversely direct the beam in any suitable manner. For example, xy-scanner 40 may include a pair of galvanometrically-actuated scanner mirrors that can be tilted about mutually perpendicular axes. As another example, xy-scanner 40 may include an electro-optical crystal that can electro-optically steer the beam or an acousto-optical crystal that can acousto-optically steer the beam. As another example, xy-scanner 40 may include a fast scanner that can create, e.g., a 3D matrix of laser pulses. In certain embodiments, xy-scanner 40 receives the xy-location of the target from imaging system 20, and directs the imaging and/or laser beam towards the xy-location.

Xy-encoder 41 detects the position of xy-scanner 40 and reports the position as the xy-location. For example, xy-encoder 41 detects the angular orientations of the galvanometer mirrors of xy-scanner 40 in encoder units. Xy-encoder 41 may report the position in encoder units to imaging system 20, laser device 22, and/or computer 26. Since imaging system 20 and laser device 22 share xy-scanner 40, computer 26 can use the encoder units to instruct system 20 and device 22 where to aim their beams, making it unnecessary to perform the computer-intensive conversion from encoder units to a length unit such as millimeters. Xy-encoder 41 may report the positions at any suitable rate, e.g., once every 5 to 50 milliseconds (ms), such as every 10 to 30 or approximately every 20 ms.

Shared components 24 also include optical elements. In general, an optical element can act on (e.g., transmit, reflect, refract, diffract, collimate, condition, shape, focus, modulate, and/or otherwise act on) a laser beam. Examples of optical elements include a lens, prism, mirror, diffractive optical element (DOE), holographic optical element (HOE), and spatial light modulator (SLM). In the example, optical elements include mirror 42 and lenses 44 and 46. Mirror 42 may be a trichroic mirror. Lenses 44 and 46 may be scanning optics of an SLO device.

Computer 26 controls components of system 10 (e.g., imaging system 20, laser device 24, and/or shared components 24) in accordance with a computer program 54. Computer 26 may be separated from components or may be distributed among system 10 in any suitable manner, e.g., within imaging system 20, laser device 24, and/or shared components 24. In certain embodiments, portions of computer 26 that control imaging system 20, laser device 24, and/or shared components 24 may be part of imaging system 20, laser device 24, and/or shared components 24, respectively.

Computer 26 controls the components of system 10 in accordance with a computer program 54. Examples of computer programs 54 include error reduction, target imaging, target tracking, and image processing programs. For example, computer 26 may use a computer program 54 to instruct imaging system 20, laser device 24, and/or shared components 24 to image a target and focus a laser beam at the target.

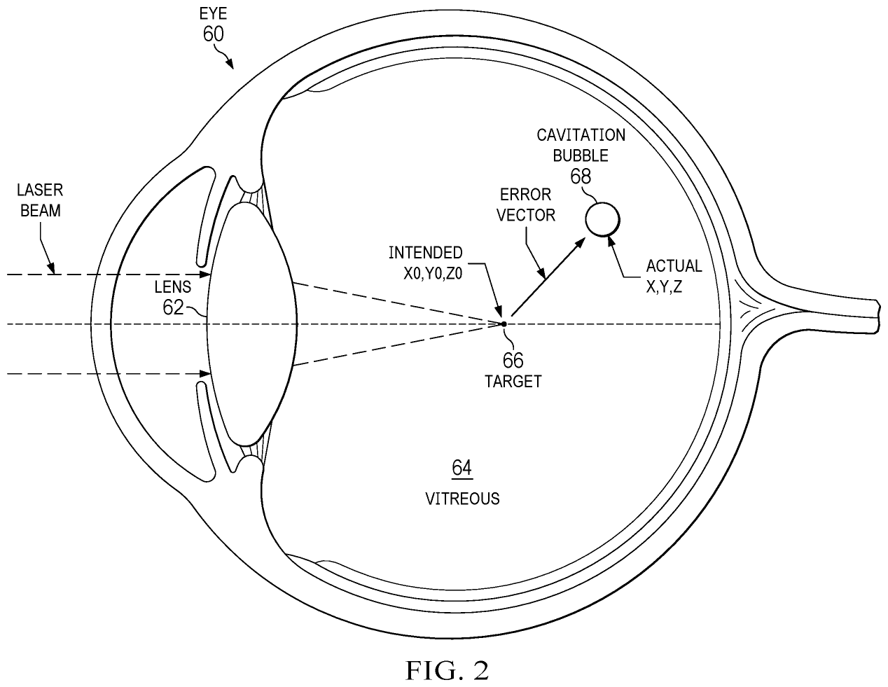

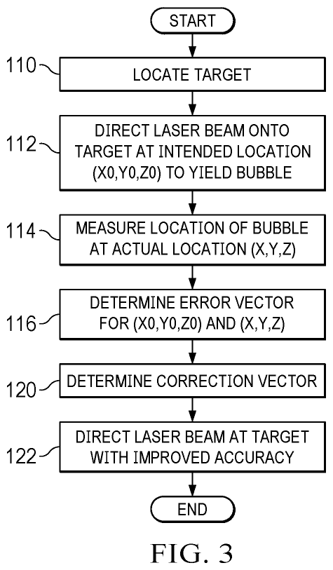

FIGS. 2 and 3 illustrate an example of a method for correcting laser beam aiming error that may be used by system 10 of FIG. 1, according to certain embodiments. FIG. 2 shows an eye 60 with a lens 62 and vitreous 64. The focus of the laser beam is directed to the intended location (x0, y0, z0) of a target 66, but due to calibration and adjustment errors of the imaging system may actually land at and form a cavitation bubble 68 at an actual location (x, y, z). The cavitation bubble may remain in vitreous 64 for several minutes if the patient is fixating his or her gaze on the fixation light of the system.

FIG. 3 shows a flowchart of the method. Steps 110 to 120 determine a calibration correction vector used to improve the aiming accuracy of the laser, and step 122 describes the treatment of the target with the calibration correction vector. At step 110, the target is located in three-dimensional (3D) space using the imaging system. The laser device directs the laser beam to the target at the intended location (x0, y0, z0) to yield a bubble at step 112. The imaging system generates a new image to measure the actual location (x, y, z) of the bubble at step 114.

The computer determines the error vector that describes the error between intended location (x0, y0, z0) and actual location (x, y, z) at step 116. In certain embodiments, the computer may calculate the error vector as (x−x0, y−y0, z−z0). The computer determines the correction vector that compensates for the error at step 120. In certain embodiments, the computer may use the opposite of the error vector as the correction vector (x0−x, y0−y, z0−z). For example, if the actual location of the bubble is 120 um below the intended location, then the laser beam should be aimed 120 um above the target appearing on the imaging system. The laser device directs the laser beam to the target with improved accuracy at step 122. Laser pulses of the beam are sent to the correct location of the target to achieve an intended surgical effect.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic laser surgical system for imaging and treating a target in an eye, comprising:
a laser device configured to:
direct a focus of a laser beam along a laser beam path towards an intended location (x0, y0, z0) of the target in a vitreous of the eye to yield a cavitation bubble in the vitreous, the eye having an eye axis, the eye axis defining a z-axis, the z-axis defining a plurality of xy-planes, an xyz-location relative to an xy-plane and the z-axis;
an imaging system configured to:
direct one or more imaging beams along an imaging beam path towards the target;
receive the one or more imaging beams reflected from the eye;
generate an image of the cavitation bubble from the reflected one or more imaging beams; and
measure an actual location (x, y, z) of the cavitation bubble according to the image; and
a computer configured to:
determine an error vector that describes an error between the intended location and the actual location;
determine a correction vector to compensate for the error; and
instruct the laser device to use the correction vector to compensate for the error to direct the laser beam towards the target to treat the target.

2. The ophthalmic laser surgical system of claim 1, the target comprising an eye floater.

3. The ophthalmic laser surgical system of claim 1, further comprising an xy-scanner configured to:
receive the one or more imaging beams from the imaging system and direct the one or more imaging beams along the imaging beam path towards the target; and
receive the laser beam from the laser device and direct the laser beam along the laser beam path aligned with the imaging beam path towards the target to treat the target.

4. The ophthalmic laser surgical system of claim 1, the error vector comprising (x−x0, y−y0, z−z0).

5. The ophthalmic laser surgical system of claim 1, the correction vector comprising (x0−x, y0−y, z0−z).

6. The ophthalmic laser surgical system of claim 1, the imaging system comprising:
an xy-imaging device configured to provide an xy-location of the target relative to an xy-plane.

7. The ophthalmic laser surgical system of claim 1, the imaging system comprising:
a z-imaging device configured to provide a z-location of the target relative to the z-axis.

8. The ophthalmic laser surgical system of claim 1, the imaging system comprising:
an xyz-imaging device configured to provide the xyz-location of the target.

9. A method for imaging and treating a target in an eye, comprising:
directing, by a laser device, a focus of a laser beam along a laser beam path towards an intended location (x0, y0, z0) of the target in a vitreous of the eye to yield a cavitation bubble in the vitreous, the eye having an eye axis, the eye axis defining a z-axis, the z-axis defining a plurality of xy-planes, an xyz-location relative to an xy-plane and the z-axis;
directing, by an imaging system, one or more imaging beams along an imaging beam path towards the target;
receiving, by the imaging system, the one or more imaging beams reflected from the eye;
generating, by the imaging system, an image of the cavitation bubble from the reflected one or more imaging beams;
measuring, by the imaging system, an actual location (x, y, z) of the cavitation bubble according to the image;
determining, by a computer, an error vector that describes an error between the intended location and the actual location;
determining, by the computer, a correction vector to compensate for the error; and
instructing, by the computer, the laser device to use the correction vector to compensate for the error to direct the laser beam towards the target to treat the target.

10. The method of claim 9, the target comprising an eye floater.

11. The method of claim 9, further comprising:
receiving, by an xy-scanner, the one or more imaging beams from the imaging system and directing the one or more imaging beams along the imaging beam path towards the target; and
receiving, by the xy-scanner, the laser beam from the laser device and directing the laser beam along the laser beam path aligned with the imaging beam path towards the target to treat the target.

12. The method of claim 9, the error vector comprising (x−x0, y−y0, z−z0).

13. The method of claim 9, the correction vector comprising (x0−x, y0−y, z0−z).

14. The method of claim 9, further comprising:
providing, by an xy-imaging device of the imaging system, an xy-location of the target relative to an xy-plane.

15. The method of claim 9, further comprising:
providing, by a z-imaging device of the imaging system, a z-location of the target relative to the z-axis.

16. The method of claim 9, further comprising:
providing, by an xyz-imaging device of the imaging system, the xyz-location of the target.
